(19) United States
(12) Patent Application Publication
(10) Pub. No.: US 2023/0143163 A1
(43) Pub. Date: May 11, 2023

(54) PROTEIN A CHROMATOGRAPHY PURIFICATION OF AN ANTIBODY

(71) Applicant: Alexion Pharmaceuticals, Inc., Boston, MA (US)

(72) Inventors: Rahul Godawat, Woodbridge, CT (US); Abraham Friedman, West Hartford, CT (US)

(73) Assignee: Alexion Pharmaceuticals, Inc., Boston, MA (US)

(21) Appl. No.: 17/920,499

(22) PCT Filed: Apr. 26, 2021
(86) PCT No.: PCT/US2021/029195

§ 371 Date: Oct. 21, 2022

Related U.S. Application Data
(60) Provisional application No. 63/016,025, filed on Apr. 27, 2020.

(51) Int. Cl. C07K 1/22; C07K 16/18

(52) U.S. Cl. CPC C07K 1/22; C07K 16/18; C07K 2317/24

(57) ABSTRACT

Disclosed are methods for purifying antibodies using a Protein A Chromatography Column and an arginine wash, resulting in decreased host cell protein (HCP) contamination. The method may comprise loading a composition comprising an antibody on a Protein A Chromatography column and washing the column with a wash solution. In an embodiment, the wash is repeated.

### CROSS-REFERENCE TO RELATED APPLICATION

This is an International Application under the Patent Cooperation Treaty, claiming priority to U.S. Provisional Patent Application No. 63/016,025 filed Apr. 27, 2020 the contents of which are incorporated herein by reference in their entirety.

### BACKGROUND

### Field

The present disclosure relates to a method of purifying an antibody using Protein A Affinity Chromatography.

### Description of Related Art

Protein A chromatography is a widely used and highly selective method relying on the strong and specific interaction between Protein A and the crystallizable fragment (Fc) of the antibody. However, host cell protein (HCP) may contaminate a final product and is preferably removed during processing. The solution to this technical problem is provided by the embodiments characterized in the claims.

### BRIEF SUMMARY

The present application relates to methods of isolating antibodies using Protein A and resulting in decreased host cell protein (HCP) contamination.

In an embodiment, method for purifying an antibody may comprise loading a composition comprising an antibody on a Protein A Chromatography column and washing the column with a wash solution. In an embodiment, the wash is repeated.

In one embodiment, a method for purifying an antibody comprising (a) Pre-culture to produce antibody expressing cells; (b) Growing antibody expressing cells in a bioreactor; (c) Harvesting the antibody by filtration; (d) Performing Protein A affinity chromatography, comprising three washes; (e) Subjecting the column to a low pH hold for viral inactivation; (f) Performing Cation Exchange Chromatography; (g) Performing Anion Exchange Chromatography; (h) Viral filtration; (i) Ultrafiltration & diafiltration; and (j) Final formulation and final filtration.

In one embodiment, the wash solution comprises a first component selected from the group consisting of tris(hydroxymethyl)aminomethane (TRIS), phosphate, acetate, arginine, propylene glycol, polysorbate 80, ethylenediaminetetraacetic acid (EDTA), sodium chloride (NaC), 3-[(3-Cholamidopropyl)dimethylammonio]-1-propanesulfonate (CHAPS), tetramethylammonium chloride (TMAC), urea, sodium caprylate, or combinations thereof. In an embodiment, the wash solution comprises a first component selected from the group consisting of TRIS, phosphate, acetate, or combinations thereof.

In one embodiment, the wash solution further comprises a second component selected from the group consisting of arginine, propylene glycol, polysorbate 80, EDTA, NaCl, CHAPS, TMAC, urea, and sodium caprylate. In an embodiment, the wash solution comprises arginine, TMAC, caprylate, or a combination thereof. In an embodiment, the wash solution comprises arginine. In an embodiment, the wash solution comprises TRIS buffer.

In one embodiment, the concentration of the first component is from about 1 mM to about 50 mM. In an embodiment, the concentration of the first component is between about 1 mM and about 50 mM, about 5 mM and about 25 mM, or about 2 mM and about 30 mM. In an embodiment, the concentration of the first component is about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or 50 mM.

In one embodiment, the concentration of the second component is from about 1 mM to about 50 M. In an embodiment, the concentration of the second component is between about 0.1 mM and 5 M, 10 mM and 1 M, 0.5 M and 2.5 M, or 0.2 M and 1 M. In an embodiment, the concentration of the second component is about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM 33 mM 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6M, 7 M, 8M, 9 M, or 10 M.

In one embodiment, the concentration of the second component is from about 0.1% and 25%. In an embodiment, the second component is between about 0.1% and 5%, 0.5% and 16%, 10% and 20%, or 1% and 25%. In an embodiment, the second component is about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

In one embodiment, the wash solution comprises arginine, propylene glycol, Polysorbate 80, EDTA, NaCl, CHAPS, TMAC, urea, sodium caprylate, Tris, phosphate, acetate, or combinations thereof, each independently in a concentration between about 0.1% and 30%. In an embodiment, each concentration is, independently, at about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

In one embodiment, the wash solution comprises arginine, propylene glycol, Polysorbate 80, EDTA, NaCl, CHAPS, TMAC, urea, sodium caprylate, Tris, phosphate, acetate, or combinations thereof, each independently in a concentration between about 1 mM and 10 M. In an embodiment, the concentration is at about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6 M, 7M, 8 M, 9 M, or 10 M.

In one embodiment, the wash solution comprises tris(hydroxymethyl)aminomethane (TRIS), phosphate, or Acetate buffer in an amount between about 1 mM and 50 mM. In an embodiment, the amount is at about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or 50 mM.

In one embodiment, the wash solution has a pH of about pH 1-11. In an embodiment, the wash solution has a pH of about pH 1, 2, 3, 4, 5, 6, 7, 8, 9, 10, or 11.

In one embodiment, the wash solution comprises 25 mM Tris and 0.5 Arginine at pH 9.

In one embodiment, the wash solution comprises 25 mM Tris and 15% propylene glycol at pH 9.

In one embodiment, the wash solution comprises 25 mM Tris and 1% polysorbate 80 at pH 9.

In one embodiment, the wash solution comprises 25 mM Tris and 20 mM EDTA at pH 9.

In one embodiment, the wash solution comprises 5 mM Tris at pH 9.

In one embodiment, the wash solution comprises 5 mM Acetate at pH 5.

In one embodiment, the wash solution comprises 25 mM Acetate and 1 M NaCl at pH 5.

In one embodiment, the wash solution comprises 25 mM Tris and 1% CHAPS at pH 9.

In one embodiment, the wash solution comprises 25 mM Tris and 0.5 TMAC at pH 9.

In one embodiment, the wash solution comprises 25 mM Tris and 1 M urea at pH 9.

In one embodiment, the wash solution comprises 25 mM and 50 mM sodium caprylate at pH 9.

In one embodiment, the method comprises three washes. In an embodiment, the first wash uses a wash solution comprising about 10-20 mM Phosphate and about 100-200 mM NaCl. In an embodiment, the second wash uses a wash solution comprising about 10-30 mM Tris and about 0.1 M to 1 M Arginine. In an embodiment, the third wash uses a wash solution comprising about 10-30 mM Acetate and about 100-200 mM NaCl.

In an embodiment, the pH of the wash solution is between about pH 4 and 1.0. In one embodiment, the pH of the wash solution is at about pH 4, pH 5, pH 6, pH 6.1, pH 6.2, pH 6.3, pH 6.4, pH 6.5, pH 6.6, pH 6.7, pH 7, pH 7.1, pH 7.2, pH 7.3, pH 7.4, pH 7.5, pH 8, pH 9, or pH 10.

In one embodiment, the washes are conducted at between about 0° C. and 50° C. In an embodiment, the washes are conducted at about 4° C.

In one embodiment, the antibody is a monoclonal, recombinant, chimeric, or humanized antibody. In an embodiment, the antibody is a fibril-reactive monoclonal antibody. In an embodiment, the antibody is humanized.

In one embodiment, the method further comprises purifying the antibody. In one embodiment, the purified antibody is substantially free of contaminants. In one embodiment, the method further comprises formulating the antibody. In an embodiment, the contaminant is host cell protein (HCP).

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows a process flow diagram for manufacturing an antibody.

FIG. 2 depicts a flowchart of an exemplary method of preparing an antibody using affinity chromatography.

FIG. 3 depicts a chromatogram control of a method to prepare an antibody, showing load, wash 1, wash 2, wash 3, elution, strip, equilibration, and cleaning. A is UV 1 280 Chrom.1:SPPD-20-0063-01C ProA Wash: B is pH Chrom.1:SPPD-20-0063-01C ProA Wash: C is Cond Chrom.1:SPPD-20-0063-01C ProA Wash.

FIG. 4 depicts a chromatogram of the method described herein to prepare an antibody including a wash with 0.5 M Arginine in place of the wash 2 shown in FIG. 3. A is UV 1 280 Chrom.1:SPPD-20-0063-01C ProA Wash; B is pH Chrom.1:SPPD-20-0063-01C ProA Wash; C is Cond Chrom.1:SPPD-20-0063-01C ProA Wash.

FIG. 5 depicts results using different “wash 2” buffers, as measured by ELISA. The trial using a 0.5 M Arginine wash showed the lowest host cell protein (HCP) contamination.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

As used herein, the term “about” modifying the quantity of an element refers to variation in the numerical quantity that can occur, for example, through typical measuring, weighing, and liquid handling procedures used for making use solutions in the real world: through inadvertent error in these procedures; through differences in the manufacture, source, or purity of the ingredients employed to make the compositions or to carry out the methods: and the like. The term “about” also encompasses amounts that differ due to different equilibrium conditions for a composition resulting from a particular initial mixture. Whether or not modified by the term “about,” the claims include equivalents to the quantities. In one embodiment, the term “about” means within 10%, preferably within 5%, more preferably within 2%, and most preferably within 1% of the reported numerical value.

### Antibody Production—Chromatography and Contaminants

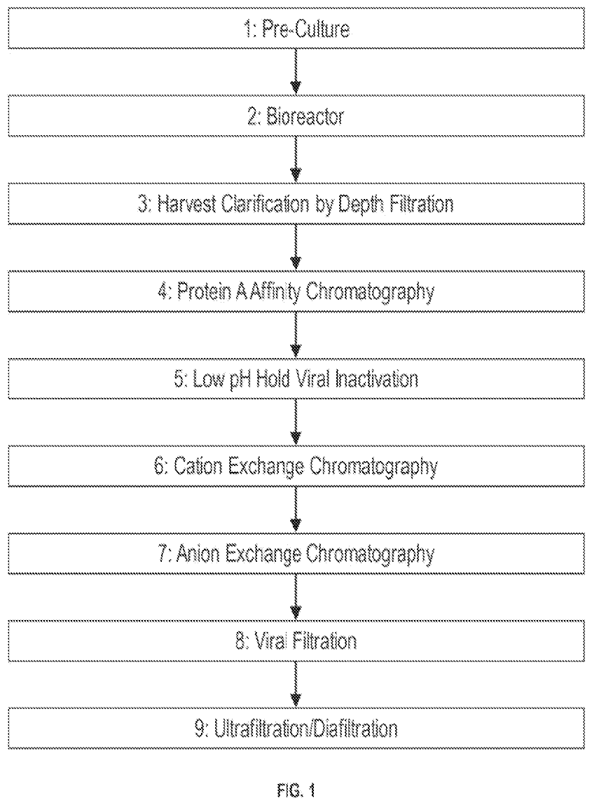

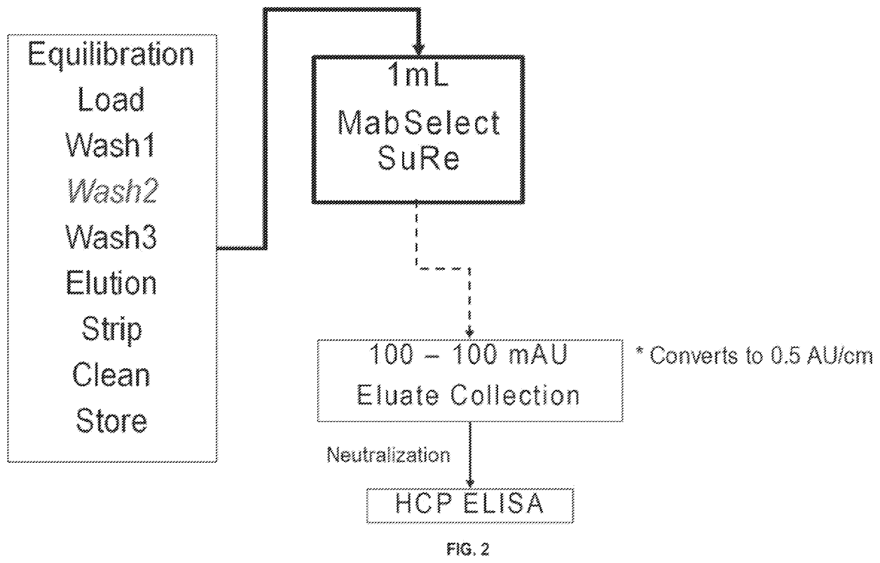

An exemplary method for preparing an antibody is outlined in FIGS. 1 and 2. The method for antibody production generally comprises culturing antibody-producing cells (including, but not limited to: Chinese hamster ovary (CHO) cells, NS0 murine myeloma cells, Sp2/0 murine myeloma cells, human embryonic kidney 293 (HEK293) cells, and PELC6® cells), collecting the antibodies from the cell culture, and loading that composition comprising the antibodies on a chromatography column to wash out contaminants, e.g., host cell proteins. Ichihara et al. (2018) MABS 10(2); 325-334. The chromatography column contains an immobile substrate to which immunoglobulin (Ig)-binding molecules have been covalently attached (immobilized). Immunoglobulin-binding molecules are known in the art and include, but are not limited to: Protein A; Protein G; Protein A/G (genetically-engineered recombinant form of Protein A and Protein G); and Protein L. Proteins A, G, and L are all bacterial proteins with well-characterized antibody-binding properties. Suitable immobile substrates for attachment to Ig-binding molecules are known in the art and include, but are not limited to: magnetic beads: beaded agarose; and polystyrene-divinylbenzene.

The antibodies in the culture medium bind to the immobilized Ig-binding molecules in the column, are washed as desired, and then the antibodies are eluted. The eluted antibodies then undergo virus inactivation and subsequent anion exchange chromatography (AEX) and cation exchange chromatography (CEX). The resultant product is then filtered, including using membrane filtration, and then final formulation of the purified antibody product. A problem that remains in the art is unacceptable amounts of host cell protein (HCP) being present in the antibody composition.

Chromatography is a widely used separation and purification technology in the preparation of antibodies. Antibody producing cells are grown in a bioreactor, and then harvested, followed by at least three unit operations, including primary capture, secondary purification, and final polishing. Several monoclonal antibody purification processes for utilize monoclonal antibody capture with a Protein A-based chromatography.

The purification and polishing steps generally incorporate cation and anion exchange chromatography, although hydrophobic interaction chromatography, mixed mode chromatography or hydroxyapatite chromatography may be used. These steps provide additional viral, host cell protein (HCP) and DNA clearance, and may remove aggregates, unwanted product variant species and other minor contaminants. Each chromatography step, either cation exchange or anion exchange, can be performed in bind and elute or flow-through mode, depending upon the physicochemical properties of the target protein and impurities.

Despite the robust nature of the chromatography, contaminants remain an issue, in particular host cell protein (HCP). The inventors surprisingly discovered that using a second wash comprising arginine during Protein A Chromatography unexpectedly and significantly decreased the amount of HCP contamination in the final product.

An exemplary method for preparing an antibody is outlined in FIGS. 1 and 2. In the Protein A Chromatography portion, three washes may be performed to remove contaminants, including host cell protein. The first wash (Wash 1) may comprise a wash solution comprising about 20 mM phosphate and about 150 mM sodium chloride (NaCl) at pH 7.2. The second wash (Wash 2) may comprise a wash solution at pH 9.0 selected from the group consisting of about 25 mM tris(hydroxymethyl)aminomethane (TRIS) and about 0.5 M Arginine; about 25 mM Tris and about 0.5 M tetramethylammonium chloride (TMAC); about 25 mM Tris and about 50 mM sodium caprylate: about 25 mM Tris and about 1% 3-[(3-Cholamidopropyl)dimethylammonio]-1-propanesulfonate (CHAPS); about 25 mM Tris and about 1% polysorbate 80 (PS80); about 25 mM Tris and about 1 M urea; and about 25 mM Tris and about 20 mM ethylenediaminetetraacetic acid (EDTA), preferably from the group consisting of about 25 mM Tris and about 0.5 M Arginine; about 25 mM Tris and about 0.5 M TMAC; and about 25 mM Tris and about 50 mM Sodium Caprylate, and most preferably about 25 mM TRIS and about 0.5 M arginine. The third wash (Wash 3) may comprise a wash solution comprising about 20 mM acetate and about 150 mM NaCl at pH 6.5. The washes may be done sequentially. The inventors surprisingly discovered that the addition and/or modification of the second wash substantially reduced host cell protein contamination.

### Wash Reagents

The wash may comprise a buffer (a first component) and a salt (a second component). The buffer for each wash solution may, independently, be tris(hydroxymethyl)aminomethane (TRIS), phosphate, or acetate buffer. The buffer may be between about 1-50 mM Tris. The buffer may be between about 1-50 mM Acetate. The buffer may be between about 1-50 mM phosphate. The concentration may be between about 1 mM and 50 mM, 5 mM and 25 mM, or 2 mM and 30 mM. The concentration may be between about 0.1 M and 5 M, 0.5 M and 2.5 M, or 0.2 M and 10 M. The concentration may be about 1 mM, 2 mM, 3 mM, 4 mM, 5 mM, 6 mM, 7 mM, 8 mM, 9 mM, 10 mM, 1l mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 125 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, or 1 M. The concentration may be about 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6 M, 7 M, 8M, 9 M, or 10 M. For example, the buffer may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM Tris; the buffer may be 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, or 30 mM Acetate; the buffer may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM phosphate.

Each wash solution may, independently, further comprise arginine, propylene glycol, polysorbate 80, EDTA, NaCl, TMAC, urea, sodium caprylate, or mixtures thereof. Each wash solution may comprise Arginine, Propylene Glycol, Polysorbate 80, EDTA, NaCl, CHAPS, TMAC, Urea, Sodium Caprylate, or a mixture thereof in a concentration between about 1 mM to 10 M. The concentration may be between about 1 mM and 50 mM, 5 mM and 25 mM, or 2 mM and 30 mM. The concentration may be between about 0.1 M and 5 M, 0.5 M and 2.5 M, or 0.2 M and 10 M. The concentration may be about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 125 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, or 1 M. The concentration may be about 0.1 M, 0.2 M, 0.3 M, 0.4 M, 0.5 M, 0.6 M, 0.7 M, 0.8 M, 0.9 M, 1 M, 2 M, 3 N¹ 4 M, 5 M, 6 M, 7 M, 8 M, 9 M, or 10 M.

Each wash solution may, independently, comprise Arginine, Propylene Glycol, Polysorbate 80, EDTA, NaCl, CHAPS, TMAC, Urea, Sodium Caprylate, or a mixture thereof in a concentration between about 0.1% to 30%. The concentration may be about 0.1%, 0.2%, 0.3%. 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The pH of the wash solution may, independently, be from about 4 to about 10. The pH may be from about 5 to about 9, about 6 to about 9, about 7 to about 9, and about 8 to about 9. The pH may be about 5, 6, 7, 8, 9, or 10. The pH may be about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0.

The wash solution may comprise 25 mM Tris and 0.5 Arginine at pH 9. The wash solution may comprise 25 mM Tris and 15% propylene glycol at pH 9. The wash solution may comprise 25 mM Tris and 1% polysorbate 80 at pH 9. The wash solution may comprise 25 mM Tris and 20 mM EDTA at pH 9. The wash solution may comprise 5 mM TRIS at pH 9. The wash solution may comprise 5 mM Acetate at pH 5. The wash solution may comprise 25 mM Acetate and 1M NaCl at pH 5. The wash solution may comprise 25 mM Tris and 1% CHAPS at pH 9. The wash solution may comprise 25 mM Tris and 0.5 TMAC at pH 9. The wash solution may comprise 25 mM Tris and 1 M urea at pH 9. The wash solution may comprise 25 mM and 50 mM sodium caprylate at pH 9.

### Example 1—CAEL-101 Purification Using a Tripe Wash Method

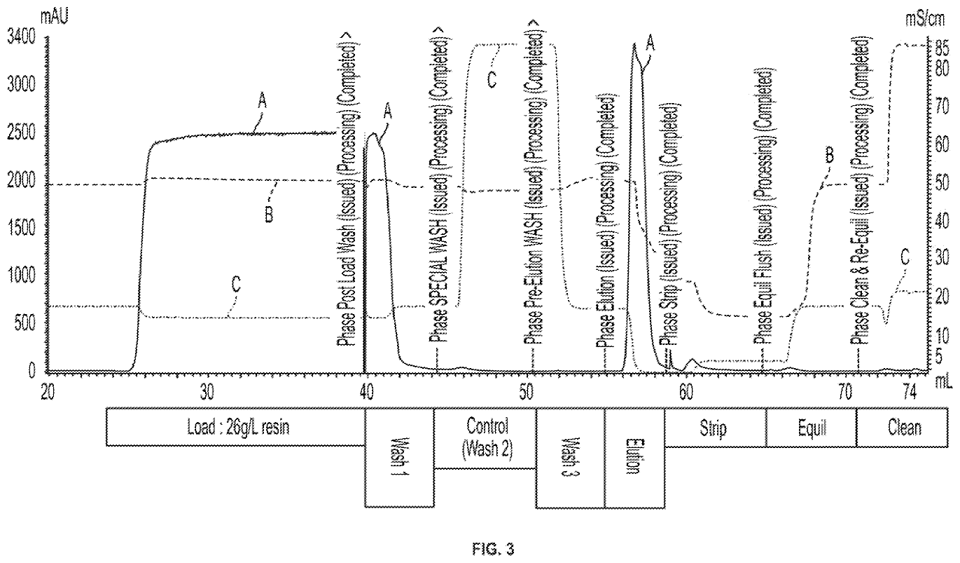

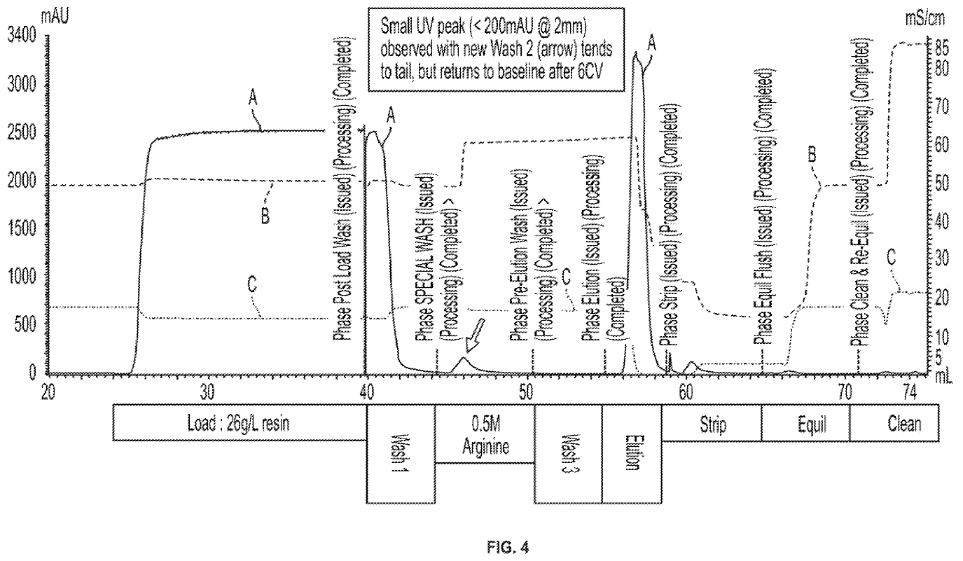

CAEL-101 antibodies (a light-chain fibril-reactive monoclonal antibody) were prepared using the method outlined in FIG. 1. The HCP contamination at each step was measured as outlined in FIGS. 3 and 4. During the purification process, a second wash was introduced into Protein A Chromatography step to improve the quality of the product. Eleven different wash solutions were tested:

1. 25 mM Tris and 0.5 Arginine at pH 9;

2. 25 mM Tris and 15% propylene glycol at pH 9:

3. 25 mM Tris and 1% polysorbate 80 at pH 9:

4. 25 mM Tris and 20 mM EDTA at pH 9;

5. 5 mM Tris at pH 9.0;

6. 5 mM Tris at pH 5;

7. 25 mM Acetate and 1M NaCl at pH 5:

8. 25 mM Tris and 1% CHAPS at pH 9:

9. 25 mM Tris and 0.5 TMAC at pH 9;

10. 25 mM Tris and 1 M urea at pH 9; and

11. 25 mM and 50 mM sodium caprylate at pH 9.

The first wash solution (FIGS. 3 & 4, “Wash 1”) comprised 20 mM Phosphate, 150 mM NaCl at pH 7.2 and the third wash solution (FIGS. 3 & 4, “Wash 3”) comprised 20 mM Acetate, 150 mM NaCl at pH 6.5.

The process performance results were measured between the 11 experimental second wash solutions and a control wash solution comprising 20 mM Phosphate, 1 M NaCl, pH 7.2.

TABLE 1
Process Performance Results

| | Resin | Elution | Elution | |
|---|---|---|---|---|
| | Load | Conc. | Volume | Recovery |
| Wash buffer | (g/L) | (mg/mL) | (g) | (%) |
| 25 mM Tris, 0.5M Arginine | 26 | 12.2 | 2.09 | 97.3 |
| pH 9.0 | | | | |
| 25 mM Tris, 15% PG, pH 9.0 | | 11.9 | 2.21 | 100.3 |
| 25 mM Tris, 1% PS80 pH 9.0 | | 11.7 | 2.21 | 98.9 |
| 25 mM Tris, 20 mM EDTA, | | 11.5 | 2.23 | 98.5 |
| pH 9.0 | | | | |
| 5 mM Tris, pH 9.0 | | 12.0 | 2.16 | 99.0 |
| 5 mM Acetate, pH 5.0 | | 11.6 | 2.14 | 95.3 |
| 25 mM Acetate, 1M NaCl, | | 12.2 | 2.10 | 98.4 |
| pH 5.0 | | | | |
| 25 mM Tris, 1% CHAPS, | | 11.4 | 2.20 | 96.4 |
| pH 9.0 | | | | |
| 25 mM Tris, 0.5M TMAC, | | 11.6 | 2.21 | 97.9 |
| pH 9.0 | | | | |
| 25 mM Tris, 1M Urea, pH 9.0 | | 11.5 | 2.22 | 97.4 |
| 25 mM Tris, 50 mM Sodium | | 11.1 | 2.29 | 97.5 |
| Caprylate, pH 9.0 | | | | |
| Control 1, 20 mM Phosphate, | | 12.5 | 2.10 | 100.5 |
| 1M NaCl, pH 7.2 | | | | |
| Control 2, 20 mM Phosphate, | | 12.2 | 2.14 | 100.3 |
| 1M NaCl, pH 7.2 | | | | |
| Control 3, 20 mM Phosphate, | | 11.9 | 2.22 | 100.9 |
| 1M NaCl, pH 7.2 | | | | |

All of the 11 experimental second wash solutions and the control solution (tested in triplicate) showed nearly identical elution concentration, elution volume, and recovery. This suggests that none of the second wash solutions adversely affects the potential recovery of the antibody.

The 11 experimental second wash solutions and the control solution were tested for the amount of HCP contamination, recovery, and log reduction value (LRV) from harvest.

TABLE 2
HCP Data

| | | Recovery * | LRV |
|---|---|---|---|
| | HCP | 1/HCP; | from |
| Buffer List | (ng/mg) | SCORE | Harvest |
| 25 mM Tris, 0.5M Arginine pH 9.0 | 888 | 13.6 | 2.218 |
| 25 mM Tris, 0.5M TMAC, pH 9.0 | 931 | 15.8 | 2.197 |
| 25 mM Tris, 50 mM Sodium Caprylate, | 1060 | 13.1 | 2.141 |
| pH 9.0 | | | |
| 25 mM Tris, 1% CHAPS, pH 9.0 | 1658 | 7.8 | 1.946 |
| 25 mM Tris, 1% PS80 pH 9.0 | 1667 | 7.1 | 1.944 |
| 25 mM Tris, 1M Urea, pH 9.0 | 1980 | 4.9 | 1.869 |
| 25 mM Tris, 20 mM EDTA, pH 9.0 | 2270 | 4.3 | 1.810 |
| 25 mM Tris, 15% PG, pH 9.0 | 3069 | 3.3 | 1.679 |
| 25 mM Acetate, 1M NaCl, pH 5.0 | 3283 | 3.0 | 1.650 |
| 5 mM Tris, pH 9.0 | 4670 | 2.1 | 1.497 |
| 5 mM Acetate, pH 5.0 | 6387 | 1.5 | 1.361 |
| Control 3, 20 mM Phosphate, 1M NaCl, | 4880 | 2.1 | 1.478 |
| pH 7.2 | | | |
| Control 2, 20 mM Phosphate, 1M NaCl, | 5161 | 1.9 | 1.453 |
| pH 7.2 | | | |
| Control 1, 20 mM Phosphate, 1M NaCl, | 6679 | 2 | 1.341 |
| pH 7.2 | | | |
| Control AVG | 5573 | | |

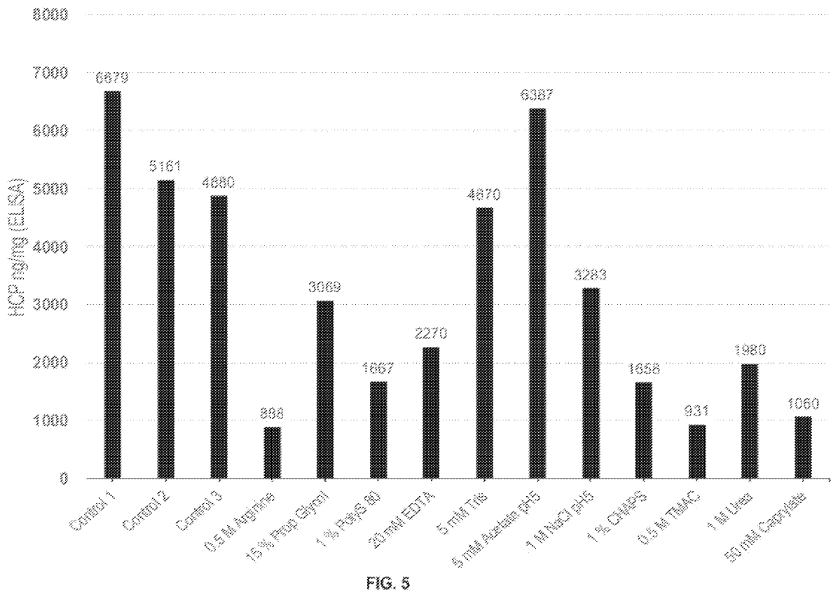

The HCP results of Table 2 are also shown in FIG. 5.

The inventors surprisingly discovered that using 25 mM Tris and 0.5 M Arginine, 25 mM Tris and 0.5 M TMAC, 25 mM Tris and 50 mM Sodium Caprylate, 25 mM Tris and 1% CHAPS, 25 mM Tris and 1% PS80, 25 mM Tris and 1 M Urea, or 25 mM Tris and 20 mM EDTA (all at pH 9.0) as a second wash solution enhanced HCP clearance by at least a factor of 2 times versus control. The inventors also surprisingly found that using 25 mM Tris and 0.5 M Arginine, 25 mM Tris and 0.5 M TMAC, or 25 mM Tris and 50 mM Sodium Caprylate (all at pH 9.0) enhanced HCP clearance by at least a factor of 5 versus control. The inventors surprisingly found that using these second wash solutions can bring HCP robustness to the antibody purification process without additional unit costs.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A method for purifying an antibody comprising loading a composition comprising an antibody on a Protein A Chromatography column and washing the column with a wash solution.

2. The method of claim 1, wherein the wash solution comprises a first component selected from the group consisting of tris(hydroxymethyl)aminomethane (TRIS), phosphate, acetate, arginine, propylene glycol, polysorbate 80, ethylenediaminetetraacetic acid (EDTA), sodium chloride (NaCl), 3-[(3-Cholamidopropyl)dimethylammonio]-1-propanesulfonate (CHAPS), tetramethylammonium chloride (TMAC), urea, sodium caprylate, or combinations thereof.

3. The method of claim 2, wherein the wash solution comprises a first component selected from the group consisting of TRIS, phosphate, acetate, or combinations thereof.

4. The method of any one of claims 1-3, wherein the wash solution further comprises a second component selected from the group consisting of arginine, propylene glycol, polysorbate 80, EDTA, NaCl, CHAPS, TMAC, urea, and sodium caprylate.

5. The method of any one of claims 1-4, wherein the wash solution comprises arginine, TMAC, caprylate, or a combination thereof.

6. The method of any one of claims 1-5, wherein the wash solution comprises arginine.

7. The method of any one of claims 1-6, wherein the wash solution comprises TRIS buffer.

8. The method of any one of claims 2-6, wherein the concentration of the first component is from about 1 mM to about 50 mM.

9. The method of claim 8, wherein the concentration of the first component is between about 1 mM and about 50 mM, about 5 mM and about 25 mM, or about 2 mM and about 30 mM.

10. The method of claim 8, wherein the concentration of the first component is about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or 50 mM.

11. The method of any one of claims 2-10, wherein the concentration of the second component is from about 1 mM to about 50 M.

12. The method of claim 11, wherein the concentration of the second component is between about 0.1 mM and 5 M, 10 mM and 1 M, 0.5 M and 2.5 M, or 0.2 M and 1 M.

13. The method of claim 11, wherein the concentration of the second component is about 1 mM, 2 mM, 3 mM, 4 mM, 5 mM, 6 mM, 7 mM, 8 mM, 9 mM, 10 mM, 1 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6 M, 7 M, 8 M, 9 M, or 10 M.

14. The method of any one of claims 2-10, wherein the concentration of the second component is from about 0.1% and 25%.

15. The method of claim 14, wherein the concentration of the second component is between about 0.1% and 5%, 0.5% and 16%, 10% and 20%, or 1% and 25%.

16. The method of claim 14, wherein the concentration of the second component is about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

17. The method of any one of claims 1-16, wherein the wash solution comprises arginine, propylene glycol, Polysorbate 80, EDTA, NaCl, CHAPS, TMAC, urea, sodium caprylate, Tris, phosphate, acetate, or combinations thereof, each independently in a concentration between about 0.1% and 3%.

18. The method of claim 17, wherein each concentration is, independently, at about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

19. The method of any one of claims 1-16, wherein the wash solution comprises arginine, propylene glycol, Polysorbate 80, EDTA, NaCl, CHAPS, TMAC, urea, sodium caprylate, Tris, phosphate, acetate, or combinations thereof, each independently in a concentration between about 1 mM and 10M14.

20. The method of claim 19, wherein the concentration is at about 1 mM, 2 mM, 3 mM, 4 mM, 5 mM, 6 mM, 7 mM, 8 mM, 9 mM, 10 mM, 1 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6M, 7 M, 8 M, 9 M, or 10 M.

21. The method of any one of claims 1-20, wherein the wash solution comprises tris(hydroxymethyl)aminomethane (TRIS), phosphate, or Acetate buffer in an amount between about 1 mM and 50 mM.

22. The method of claim 21, wherein the amount is at about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM 47 mM, 48 mM, 49 mM, or 50 mM.

23. The method of any one of claims 1-22, wherein the wash solution has a pH of about pH 1-11.

24. The method of any one of claims 1-23, wherein the wash solution has a pH of about pH 1, 2, 3, 4, 5, 6, 7, 8, 9, 10, or 11.

25. The method of any one of claims 1-24, wherein the wash solution comprises 25 mM Tris and 0.5 Arginine at pH 9.

26. The method of any one of claims 1-24, wherein the wash solution comprises 25 mM Tris and 15% propylene glycol at pH 9.

27. The method of any one of claims 1-24, wherein the wash solution comprises 25 mM Tris and 1% polysorbate 80 at pH 9.

28. The method of any one of claims 1-24, wherein the wash solution comprises 25 mM Tris and 20 mM EDTA at pH 9.

29. The method of any one of claims 1-24, wherein the wash solution comprises 5 mM Tris at pH 9.

30. The method of any one of claims 1-24, wherein the wash solution comprises 5 mM Acetate at pH 5.

31. The method of any one of claims 1-24, wherein the wash solution comprises 25 mM Acetate and 1M NaCl at pH 5.

32. The method of any one of claims 1-24, wherein the wash solution comprises 25 mM Tris and 1% CHAPS at pH 9.

33. The method of any one of claims 1-24, wherein the wash solution comprises 25 mM Tris and 0.5 TMAC at pH 9.

34. The method of any one of claims 1-24, wherein the wash solution comprises 25 mM Tris and 1 M urea at pH 9.

35. The method of any one of claims 1-24, wherein the wash solution comprises 25 mM and 50 mM sodium caprylate at pH 9.

36. The method of any one of claims 1-35, wherein the method comprises three washes.

37. The method of claim 36, wherein the first wash uses a wash solution comprising about 10-20 mM Phosphate and about 100-20 mM NaCl.

38. The method of claim 36, wherein the second wash uses a wash solution comprising about 10-30 mM Tris and about 0.1 M to 1 M Arginine.

39. The method of claim 36, wherein the third wash uses a wash solution comprising about 10-30 mM Acetate and about 100-200 mM NaCl.

40. The method of any one of claims 36-39, wherein the pH of the wash solution is between about pH 4 and 10.

41. The method of claim 40, wherein the pH of the wash solution is at about pH 4, pH 5, pH 6, pH 6.1, pH 6.2, pH 6.3, pH 6.4, pH 6.5, pH 6.6, pH 6.7, pH 7, pH 7.1, pH 7.2, pH 7.3, pH 7.4, pH 7.5, pH 8, pH 9, or pH 10.

42. The method of any one of claims 1-41, wherein the washes are conducted at between about 0° C. and 50° C.

43. The method of any one of claims 1-42, wherein the antibody is a monoclonal, recombinant, chimeric, or humanized antibody.

44. The method of any one of claims 1-42, wherein the antibody is a fibril-reactive monoclonal antibody.

45. The method of any one of claims 1-42, wherein the antibody is humanized.

46. The method of any one of claims 1-42, further comprising purifying the antibody.

47. The method of claim 46, further comprising formulating the antibody.

48. The method of claim 46, wherein the purified antibody is substantially free of contaminants.

49. The method of claim 48, wherein the contaminant is host cell protein (HCP).

50. The method of any one of claims 1-49, wherein the wash step is repeated.

51. A method for purifying an antibody comprising
(a) Pre-culture to produce antibody expressing cells;
(b) Growing antibody expressing cells in a bioreactor;
(c) Harvesting the antibody by filtration;
(d) Performing Protein A affinity chromatography, comprising three washes;
(e) Subjecting the column to a low pH hold for viral inactivation;
(f) Performing Cation Exchange Chromatography;
(g) Performing Anion Exchange Chromatography;
(h) Viral filtration;
(i) Ultrafiltration & diafiltration; and
(j) Final formulation and final filtration.

52. The method of claim 51, wherein the wash solution comprises a first component selected from the group consisting of tris(hydroxymethyl)aminomethane (TRIS), phosphate, acetate, arginine, propylene glycol, polysorbate 80, ethylenediaminetetraacetic acid (EDTA), sodium chloride (NaCl), 3-((3-Cholamidopropyl)dimethylammonio-1-propanesulfonate (CHAPS), tetramethylammonium chloride (TMAC), urea, sodium caprylate, or combinations thereof.

53. The method of claim 52, wherein the wash solution comprises a first component selected from the group consisting of TRIS, phosphate, acetate, or combinations thereof.

54. The method of any one of claims 51-53, wherein the wash solution further comprises a second component selected from the group consisting of arginine, propylene glycol, polysorbate 80, EDTA, NaCl, CHAPS, TMAC, urea, and sodium caprylate.

55. The method of any one of claims 51-54, wherein the wash solution comprises arginine, TMAC, caprylate, or a combination thereof.

56. The method of any one of claims 51-55, wherein the wash solution comprises arginine.

57. The method of any one of claims 51-56, wherein the wash solution comprises TRIS buffer.

58. The method of any one of claims 52-56, wherein the concentration of the first component is from about 1 mM to about 50 mM.

59. The method of claim 58, wherein the concentration of the first component is between about 1 mM and about 50 mM, about 5 mM and about 25 mM, or about 2 mM and about 30 mM.

60. The method of claim 58, wherein the concentration of the first component is about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or 50 mM.

61. The method of any one of claims 52-60, wherein the concentration of the second component is from about 1 mM to about 50 M.

62. The method of claim 61, wherein the concentration of the second component is between about 0.1 mM and 5 M, 10 mM and 1 M, 0.5 M and 2.5 M, or 0.2 M and 1 M.

63. The method of claim 61, wherein the concentration of the second component is about 1 mM, 2 mM, 3 mM, 4 mM, 5 mM, 6 mM, 7 mM, 8 mM, 9 mM, 10 mM, I1 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6 M, 7 M, 8 M, 9 M, or 10 M.

64. The method of any one of claims 52-60, wherein the concentration of the second component is from about 0.1% and 25%.

65. The method of claim 64, wherein the concentration of the second component is between about 0.1% and 5%, 0.5% and 16%, 10% and 20%, or 1% and 25%.

66. The method of claim 64, wherein the concentration of the second component is about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

67. The method of any one of claims 51-66, wherein the wash solution comprises arginine, propylene glycol, Polysorbate 80, EDTA, NaCl, CHAPS, TMAC, urea, sodium caprylate, Tris, phosphate, acetate, or combinations thereof, each independently in a concentration between about 0.1% and 30%.

68. The method of claim 67, wherein each concentration is, independently, at about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

69. The method of any one of claims 51-68, wherein the wash solution comprises arginine, propylene glycol, Polysorbate 80, EDTA, NaCl, CHAPS, TMAC, urea, sodium caprylate, Tris, phosphate, acetate, or combinations thereof, each independently in a concentration between about 1 mM and 10M.

70. The method of claim 69, wherein the concentration is at about 1 mM, 2 mM, 3 mM, 4 mM, 5 mM, 6 mM, 7 mM, 8 mM, 9 mM, 10 mM, 11 mM, 12 mM, 13 mM, 14 mM 15 mM 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 0.1 M, 0.2 M, 0.3 M, 0.4 M, 0.5 M, 0.6 M, 0.7 M, 0.8 M, 0.9 M, 1 M, 2 M, 3 M, 4 M, 5 M, 6 M, 7 M, 8 M9M, or 10 M.

71. The method of any one of claims 51-70, wherein the wash solution comprises tris(hydroxymethyl)aminomethane (TRIS), phosphate, or Acetate buffer in an amount between about 1 mM and 50 mM.

72. The method of claim 71, wherein the amount is at about 1 mM, 2 mM, 3 mM, 4 mM, 5 mM, 6 mM, 7 mM, 8 mM, 9 mM, 10 mM, I1 mM, 12 mM 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM 47 mM, 48 mM, 49 mM, or 50 mM.

73. The method of any one of claims 51-72, wherein the wash solution has a pH of about pH 1-11.

74. The method of any one of claims 51-73, wherein the wash solution has a pH of about pH 1, 2, 3, 4, 5, 6, 7, 8, 9, 10, or 11.

75. The method of any one of claims 51-74, wherein the wash solution comprises 25 mM Tris and 0.5 Arginine at pH 9.

76. The method of any one of claims 51-74, wherein the wash solution comprises 25 mM Tris and 15% propylene glycol at pH 9.

77. The method of any one of claims 51-74, wherein the wash solution comprises 25 mM Tris and 1% polysorbate 80 at pH 9.

78. The method of any one of claims 51-74, wherein the wash solution comprises 25 mM Tris and 20 mM EDTA at pH 9.

79. The method of any one of claims 51-74, wherein the wash solution comprises 5 mM Tris at pH 9.

80. The method of any one of claims 51-74, wherein the wash solution comprises 5 mM Acetate at pH 5.

81. The method of any one of claims 51-74, wherein the wash solution comprises 25 mM Acetate and 1M NaCl at pH 5.

82. The method of any one of claims 51-74, wherein the wash solution comprises 25 mM Tris and 1% CHAPS at pH 9.

83. The method of any one of claims 51-74, wherein the wash solution comprises 25 mM Tris and 0.5 TMAC at pH 9.

84. The method of any one of claims 51-74, wherein the wash solution comprises 25 mM Tris and 1 M urea at pH 9.

85. The method of any one of claims 51-74, wherein the wash solution comprises 25 mM and 50 mM sodium caprylate at pH 9.

86. The method of any one of claims 51-85, wherein the method comprises three washes.

87. The method of claim 86, wherein the first wash uses a wash solution comprising about 10-20 mM Phosphate and about 100-200 mM NaCl.

88. The method of claim 86, wherein the second wash uses a wash solution comprising about 10-30 mM Tris and about 0.1 M to 1 M Arginine.

89. The method of claim 86, wherein the third wash uses a wash solution comprising about 10-30 mM Acetate and about 100-200 mM NaCl.

90. The method of any one of claims 86-89, wherein the pH of the wash solution is between about pH 4 and 10.

91. The method of claim 90, wherein the pH of the wash solution is at about pH 4, pH 5, pH 6, pH 6.1, pH 6.2, pH 6.3, pH 6.4, pH 6.5, pH 6.6, pH 6.7, pH 7, pH 7.1, pH 7.2, pH 7.3, pH 7.4, pH 7.5, pH 8, pH 9, or pH 10.

92. The method of any one of claims 51-91, wherein the washes are conducted at between about 0° C. and 50° C.

93. The method of any one of claims 51-92, wherein the antibody is a monoclonal, recombinant, chimeric, or humanized antibody.

94. The method of any one of claims 51-92, wherein the antibody is a fibril-reactive monoclonal antibody.

95. The method of any one of claims 51-92, wherein the antibody is humanized.

96. The method of any one of claims 51-92, further comprising purifying the antibody.

97. The method of claim 96, further comprising formulating the antibody.

98. The method of claim 96, wherein the purified antibody is substantially free of contaminants.

99. The method of claim 98, wherein the contaminant is host cell protein (HCP).

100. The method of any one of claims 1-99, wherein the wash step is repeated.
